SEQUENCE LISTING (10 sequences)

SEQ ID NO: 1 - DNA, 418 bp, Felis catus
ctgcatcatgaagggggctcgtgttctcgtgcttctctgggctgccttgctcttgatctggggtggaaattgtgaaatttgcccagccgtgaagagggatgttgacctattcctgacgggaacccccgacgaatatgttgagcaagtggcacaatacaaagcactacctgtagtattggaaaatgccagaatactgaagaactgcgttgatgcaaaaatgacagaagaggataaggagaatgctctcagcttgctggacaaaatatacacaagtcctctgtgttaaaggagccatcactgccaggagccctaaggaagccactgaactgatcactaagtagtctcagcagcctgccatgtccaggtgtcttactagaggattccagcaataaaagcctggcaattcaaacaaaaaaaa

SEQ ID NO: 2 - protein, 94 aa, Felis catus
CIMKGARVLVLLWAALLLIWGGNCEICPAVKRDVDLFLTGTPDEYVEQVAQYKALPVVLENARILKNCVDAKMTEEDKENALSLLDKIYTSPLC

SEQ ID NO: 3 - DNA, 420 bp, Felis catus
ggcctggcggtgctcctggaaaaggatgttagacgcagccctcccaccctgccctactgttgcggccacagcagattgtgaaatttgcccagccgtgaagagggatgttgacctattcctgacgggaacccccgacgaatatgttgagcaagtggcacaatacaaagcactacctgtagtattggaaaatgccagaatactgaagaactgcgttgatgcaaaaatgacagaagaggataaggagaatgctctcagcttgctggacaaaatatacacaagtcctctgtgttaaaggagccatcactgccaggagccctaaggaagccactgaactgatcactaagtagtctcagcagcctgccatgtccaggtgtcttactagaggattccagcaataaaagccttgcaattcaaacaaaa

SEQ ID NO: 4 - protein, 96 aa, Felis catus
AWRCSWKRMLDAALPPCPTVAATADCEICPAVKRDVDLFLTGTPDEYVEQVAQYKALPVVLENARILKNCVDAKMTEEDKENALSLLDKIYTSPLC

SEQ ID NO: 5 - DNA, 476 bp, Felis catus
tgacacgatgaggggggcactgcttgtgctggcattgctggtgacccaagcgctgggcgtcaagatggcggaaacttgccccattttttatgacgtcttttttgcggtggccaatggaaatgaattactgttggacttgtccctcacaaaagtcaatgctactgaaccagagagaacagccatgaaaaaaatccaggattgctacgtggagaacggactcatatccagggtcttggatggactagtcatgacaaccatcagctccagcaaagattgcatgggtgaagcagttcagaacaccgtagaagatctcaagctgaacactttggggagatgaatctttgccactgatgccccttctgagccccatcctcctgccctgttctttacacctaaagctggaatccagacacctgtcctcacctaattcactctcaatcaggctgactagaataaaataactgcatcttaaaaaa

SEQ ID NO: 6 - protein, 92 aa, Felis catus
VKMAETCPIFYDVFFAVANGNELLLDLSLTKVNATEPERTAMKKIQDCYVENGLISRVLDGLVMTTISSSKDCMGEAVQNTVEDLKLNTLGR

SEQ ID NO: 7 - DNA, 469 bp, Felis catus
gacacgatgaggggggcactgcttgtgctggcattgctggtgacccaagcgctgggcgtcaagatggcggagacgtgccccattttttatgacgtcttttttgcggtggccaatggaaatgaattactgttggacttgtccctcacaaaagtcaatgctactgaaccagagagaacagccatgaaaaaaatccaggattgctacgtggagaacggactcatatccagggtcttggatggactagtcatgatagccatcaacgaatattgcatgggtgaagcagttcagaacaccgtagaagatctcaagctgaacactttggggagatgaatctttgccactgatgccccttctgagccccatcctcctgtcctgttctttacacctaaagctggaatccagacacctgtcctcacctaattcactctcaatcaggctgactagaataaaataactgcatcttaaaaaa

SEQ ID NO: 8 - protein, 90 aa, Felis catus
VKMAETCPIFYDVFFAVANGNELLLDLSLTKVNATEPERTAMKKIQDCYVENGLISRVLDGLVMIAINEYCMGEAVQNTVEDLKLNTLGR

SEQ ID NO: 9 - DNA, 465 bp, Felis catus
gacacgatgaggggggcactgcttgtgctggcattgctggtgacccaagcgctgggcgtcaagatggcggagacgtgccccattttttatgacgtcttttttgcggtggccaatggaaatgaattactgttggacttgtccctcacaaaagtcaatgctactgaaccagagagaacagccatgaaaaaaatccaggattgctacgtggagaacggactcatatccagggtcttggatggactagccatgccatcaacgaatattgcatgggtgaagcagttcagaacaccgtagaagatctcaagctgaacactttggggagatgaatctttgccactgatgccccttctgagccccatcctcctgtcctgttctttacacctaaagctggaatccagacacctgtcctcacctaattcactctcaatcaggctgactagaataaaataactgcatcttaaaaaa

SEQ ID NO: 10 - protein, 51 aa, Felis catus
SLTKVNATEPERTAMKKIQDCYVENGLISRVLDGLVMPSTNIAWVKQFRTP

(19) United States
(12) Patent Application Publication
(10) Pub. No.: US 2007/0231341 A1
(43) Pub. Date: Oct. 4, 2007

(54) Cat dander allergen treatments

(76) Inventors: David R. McGavin, Portage, MI (US); John David Haworth, Kalamazoo, MI (US); Rajendra Krishnan, Portage, MI (US); John M. Hardham, Kalamazoo, MI (US)

Correspondence Address: PHARMACIA & UPJOHN, 7000 Portage Road, KZO-300-104, KALAMAZOO, MI, 49001, US

(21) Appl. No.: 11/731,555

(22) Filed: Mar. 30, 2007

Related U.S. Application Data
(60) Provisional application No. 60/788,798, filed on Apr. 3, 2006.

(51) Int. Cl. A61K 48/00; A61K 39/00

(52) U.S. Cl. 424/185.1; 514/44

(57) ABSTRACT

The present invention provides a method of reducing the major allergen in cat dander, Fel d1, and of reducing allergic response in mammals, including humans, sensitive to cat dander. Specifically to humans sensitive to the Fel d1 allergen that is shed by a cat. The treatment is achieved through administering to the cat itself a composition, which stimulates the cat's immune response to its own dander and Fel d1 polypeptide. The result is a reduction in the amount of Fel d1 shed by the cat, and a subsequent reduction or lowering of the level of allergic responsiveness in sensitized individuals.

### CROSS-REFERENCE TO RELATED APPLICATIONS

This application claims the benefit of U.S. provisional patent application U.S. Ser. No. 60/788,798, filed on Apr. 3, 2006, under 35 USC 119(e)(i), incorporated herewith in its entirety.

### BACKGROUND

There are approximately 10 million people in the United States who are allergic to cats. Ohman, J. L., and Sundin, B., Clin. Rev. Allergy, 5:37-47 (1987). An allergen of particular concern for many is the feline skin and salivary gland allergen of the domestic cat Felis domesticus allergen I, herein Fel d1, is also referred to as: allergen I, cat 1 and antigen 4. Exposure to this allergen elicits an IgE-mediated allergic response in sensitized individuals.

Current treatment methods for humans with respect to this and other allergic IgE-mediated diseases employ agents which provide symptomatic relief, prevention or desensitization. Examples of such agents which provide symptomatic relief and prevention are anti-histamines, β₂ agonists, and glucocorticosteroids. Desensitization procedures, often directed to IgE-mediated diseases, involve the periodic injection of the sensitized host with allergen components or extracts. Desensitization treatments may induce an IgG response that competes with IgE for allergen, or they may induce specific suppressor T cells that block the synthesis of IgE directed against allergen. This form of treatment is not always effective and poses the risk of provoking serious side effects, particularly general anaphylactic shock, which can be fatal.

The present invention provides a better and safer approach to treating Fel d1 allergies in sensitized individuals. Here we describe a novel method of helping or treating humans who are allergic to cats. We describe methods to make felines less allergic to themselves and other mammals.

### DETAILED DESCRIPTION OF THE INVENTION

### SUMMARY OF THE INVENTION

The invention comprises new uses for various polypeptides, nucleotides, methods of administration and new treatments. We provide for methods of reducing the amount of Fel d1 shed by a cat comprising: administering to a cat an immunogenic composition comprising at least one Fel d1 polypeptide or fragment thereof, or a polynucleotide molecule encoding one Fel d1 polypeptide or fragment thereof, or a viral vector containing a Fel d1 nucleotide or fragment, or a Fel d1 polypeptide or fragment of at least one recombinantly-produced Fel d1 polypeptide or fragment, or a polynucleotide molecule encoding such a polypeptide or fragment, and/or at least one naturally-occurring Fel d1 polypeptide or fragment including such polypeptide or fragment conjugated to a carrier polypeptide, including a heterologous carrier polypeptide, and polynucleotide molecule encoding such a carrier all optionally acting in association with pharmaceutically-acceptable carriers.

We describe a method of reducing the amount of Fel d1 shed by a cat comprising: 1) administering to a cat immunogenic compositions comprising monoclonal antibodies raised against Fel d1 and or 2) using RNA silencing, i.e. siRNA methods where double stranded RNAs (dsRNAs) are used to silence expression of the Fel d1 polynucleotides of fragments thereof, using full length or fragments of dsRNAs.

We describe the compositions above administered to a cat when they are administered to a cat more than once, twice, orally, administered by parenteral, subcutaneous, and intramuscular injection.

We describe the compositions above administered and used to treat mammals who are allergic to cats, especially for treatments of humans, cats (both self and other), dogs and horses.

### BRIEF DESCRIPTION OF THE SEQUENCE LISTING

SEQ. ID. NO: 1. The DNA sequence encoding Fel d1, chain 1, Leader A.

SEQ. ID. NO: 2. The amino acid sequence of Fel d1, chain 1, Leader A.

SEQ. ID. NO: 3. The DNA sequence encoding Fel d1, chain 1, Leader B.

SEQ. ID. NO: 4. The amino acid sequence of Fel d1, chain 1, Leader B.

SEQ. ID. NO: 5. The DNA sequence encoding Fel d1, chain 2, Long form.

SEQ. ID. NO: 6. The amino acid sequence of Fel d1, chain 2, Long form.

SEQ. ID. NO: 7. The DNA sequence encoding Fel d1, chain 2, Short form.

SEQ. ID. NO: 8. The amino acid sequence of Fel d1, chain 2, Short form.

SEQ. ID. NO: 9. The DNA sequence encoding Fel d1, chain 2, Short Truncated form.

SEQ. ID. NO: 10. The amino acid sequence of Fel d1, chain 2, Short Truncated form.

### DEFINITIONS

“Antigen” is any substance that may provoke an immune response or that may be recognized by the immune system of an individual.

“Carrier(s)” of Fel d1, are any protein, peptide, polypeptides or fragments thereof, which act to provoke or induce the immune system to respond to Fel d1, in either a direct or indirect manner. Carrier(s) can also refer to a virus-like particle (VLP). A carrier could be a biological nano designed structure. A carrier is not an adjuvant per se, but it can act like an adjuvant, that is, it may boost or increase an immune response to a given antigen. An indirect carrier example is egg albumen given to increase the immune response to Fel d1, while a more direct carrier might be a protein joined or fused in some manner to all or part of Fel d1. A joined protein could be a fusion protein; it could be a heterologous polypeptide, or a recombinant polypeptide. A fused or a heterologous polypeptide may be joined (“conjugated”) to the antigenic polypeptide, either through chemical fixation, via recombinant technology, or other techniques known to those of skill in the art.

“Cat” is any member of the family Felidae, including Felis domesticus, or the common house cat.

“Cat dander” see Fel d1 peptide.

“Cat protein allergen” see Fel d1 peptide.

“Fel d1” is the cat protein allergen Felis domesticus allergen 1, also known as “human T cell reactive feline protein” (TRFP), i.e. a feline protein to which human T cells are reactive, it has also been referred to as “allergen 1”, “cat 1” and “antigen 4”, or for purposes here when used by itself, may also refer to the nucleic acids that encode for the protein, or any peptide or DNA fragments or components thereof, whether alone, isolated or in combination, or conjugated with other peptides, DNA or carriers. Fel d1 is described in U.S. Pat. No. 6,048,962, col. 6, lines 28-38 and lines 51-54, herein incorporated by reference.

The description in U.S. Pat. No. 6,048,962 of Fel d1 is as follows. In U.S. Pat. No. 6,049,962 the term TRFP is used instead of Fel d1, The cat protein allergen, human T cell reactive feline protein (TRFP) has been isolated and purified by affinity purification of vacuum cleaner bag house dust collected from several homes with cats. “Isolated” refers to the TRFP protein or peptides free of all other cat polypeptides or contaminants. The work described herein has resulted in isolation and purification of a TRFP protein; the nucleotide sequence encoding TRFP and the amino acid sequence of TRFP has been determined. TRFP is composed of two covalently linked peptide chains (designated chain 1 and 2). Chain 1 of the two-chain TRFP protein has two alternative leader sequences and that chain 2 has two major forms (designated as long and short).

“Fel d1 peptide or peptides” is either one or both peptide chains of Fel d1, as well as immunogenic fragments thereof, see Fel d1. Native Fel d1 peptide is composed of two covalently linked peptide chains, described here as chain 1 and chain 2. Chain 1 of the two-chain Fel d1 protein can possess one of two alternative leader sequences, while chain 2 can be isolated in one of three forms. These sequences are described in the Brief Description of the Sequence Listings and the Sequence Listing itself.

“Fel d1 nucleotides” are the nucleotides that encode for any region of the Fel d1 proteins, polypeptide(s) or peptide fragments. These sequences are described in the Brief Description of the Sequence Listings and the Sequence Listing itself.

“Isolated Fel d1” is Fel d1 that is substantially free of other cat polypeptides or nucleic acids.

“Feline” is any member of the family Felidae, including Felis domesticus, or the common house cat.

“Immunogenic composition” is a composition that generates an immune response (i.e., has immunogenic activity) when administered alone or with a pharmaceutically acceptable carrier, to an animal, such as a mammal, including a cat.

“Naturally occuring” or “native” is a polypeptide derived from its natural host; i.e. not recombinantly produced.

“Orally”, “oral” or “oral administration” means the introduction of a substance, such as a vaccine, into a subject's body through or by way of the mouth and involves swallowing or transport through the oral mucosa (e.g., sublingual or buccal absorption) or both. Oral includes all administration routes that primarily involve transport of the substance through mucosal tissue in the mouth, nose, trachea, and lungs.

“Parenterally”, “parenteral”, or “parenteral administration,” means the introduction of a substance, such as a vaccine, into a subject's body through or by way of a route that does not include the digestive tract. Parenteral administration includes subcutaneous administration, intramuscular administration, transcutaneous administration, intradermal administration, intraperitoneal administration, intraocular administration, and intravenous administration.

“Pharmaceutically acceptable carrier” is a carrier medium that does not interfere with the effectiveness of the biological activity of the active ingredient, and is not toxic to the subject to whom it is administered.

“Recombinantly produced” or “recombinant” is a polypeptide produced outside of its natural host; i.e. not naturally occurring or native protein. Recombinantly produced polypeptide can be produced in, but is not limited to, bacterial, viral, yeast expression, or artificial chromosome systems.

“Reducing” or “reduction” with reference to Fel d1, refers to a lowering or decrease in the level of Fel d1 polypeptide present or produced, including but not limited to elimination, on the external surface of a feline, or in the area that felines inhabit. “Reduce,” “reducing” or “reduction” as used herein with reference to mammalian allergic responsiveness, refers to a lowering or decrease in the level or severity of the immune response generated by a mammal upon exposure to Fel d1.

“Sensitivity” is the ability to generate an IgE-mediated immune response upon exposure to an allergen.

“Shed” is the release of Fel d1 polypeptide from, for example the sebaceous glands, onto the external surface of a cat's body. The polypeptide can subsequently remain associated with the skin or hair, or can be released into the environment.

“Treatment” is any method to reduce the suffering or symptoms of a susceptible individual. It may describe prophylactic administration of a composition and it may describe active steps to change the individual's environment, such as reducing the exposure to an antigen.

“Viral vector”, is a virus-based tool that allows or facilitates the transfer of a nucleic acid from one environment to another. Viral vectors allow nucleic acids, such as a segment of DNA (such as a heterologous DNA segment), to be transferred into a host or a target cell for the purpose of replicating the nucleic acids and/or expressing proteins encoded by the nucleic acids.

1) Fel d1 Peptides and Methods of Administration.

The present invention provides a method of reducing the amount of Fel d1 shed by a cat. The cat is administered an immunogenic composition that comprises a Fel d1 polypeptide or immunogentic fragment thereof. A member of the genus Felidae, typically a domestic cat, is administered an immunogenic composition comprising at least one whole or a fragment of a Fel d1 polypeptide. This includes the administration of an immunogenic composition to a cat which comprises at least one complete or partial Fel d1 polypeptide, native, recombinant, optionally provided with a carrier protein.

Fel d1 is composed of two covalently linked peptide chains, designated Chain 1 and Chain 2. Chain 1 with leader sequence (Leader) A (SEQ ID NO. 2) is dominant in both salivary glands and skin. Chain 1 with leader sequence (Leader) B (SEQ ID NO. 4) is a minor component of both the salivary glands and skin. Three forms of Chain 2 (Long, Short, and Short Truncated) have been isolated from the salivary glands and skin of cats. Chain 2 Long form (SEQ ID NO. 6) is the dominant form in the salivary glands. Chain 2 Short form (SEQ ID NO. 8) is dominant in the skin, while the Long and the Short Truncated forms (SEQ ID NO. 10) are minor forms.

Also disclosed are smaller fragments of Fel d1 polypeptide that are known to provoke an immune response. These fragments of Fel d1 polypeptide can be easily determined by one skilled in the art using the Examples provided herein.

Native Fel d1 Polypeptide.

In an alternate embodiment, the immunogenic composition contains a naturally-occurring Fel d1 polypeptide that can be isolated directly or indirectly from cats. Fel d1 may be extracted from cat hair or squames, from cat saliva, from other parts of the cat or other environmental sources such as dust from areas where cats are present, by any of a number of methods commonly known to those of skill in the art, including surface washing of cats, affinity purification using polyclonal or monoclonal antibodies, and other biochemical methods.

A cat may be administered an immunogenic composition comprising at least one Fel d1 polypeptide obtained from its natural environment.

Carriers of Fel d1.

“Carriers of Fel d1”, as used herein, refers to any substance which acts to provoke or induce the immune system to respond to Fel d1, in either a direct or indirect manner. More typically it refers to a protein, peptide, polypeptides or fragments thereof, which act to provoke or induce the immune system to respond to Fel d1. It can also refer to a VLP. It could refer to an artificial structure such as biological nano designed structure. An indirect carrier might be something like egg albumen given to increase the immune response to Fel d1, while a more direct carrier might be a protein joined or fused in some manner to all or part of Fel d1. Immunogenic carrier proteins include but are not limited to, intact Keyhole Limpet Hemocyanin (KLH) subunit KLH, or Diptheria Toxoid (DT).

A joined protein could be a fusion protein, it could be a heterologous polypeptide, or a recombinate polypeptide. A fused or a heterologous polypeptide may be joined (“conjugated”) to the antigenic polypeptide, either through chemical fixation, via recombinant technology, or other techniques known to those of skill in the art. Further details and possible “carriers” are described below.

Recombinant Fel d1 Polypeptide.

In a further embodiment, the Fel d1 polypeptide present in the immunogenic composition can be recombinantly produced.

Recombinant Fel d1 of the present invention may be produced via a variety of host-expression vector systems. Such host-expression vector systems include, but are not limited to, microorganisms such as bacteria (e.g. Escherichia coli, Bacillus subtilis) transformed with recombinant bacteriophage DNA, plasmid DNA or cosmid DNA expression vectors containing coding sequences, yeast (e.g., Saccharomyces, Pichia) transformed with recombinant yeast expression vectors containing the gene product coding sequences; insect cell systems infected with recombinant virus expression vectors (e.g., baculovirus) containing the coding sequences, plant cell systems infected with recombinant virus expression vectors (e.g., cauliflower mosaic virus or tobacco mosaic virus) or transformed with recombinant plasmid expression vectors (e.g., Ti plasmid) containing coding sequences, or mammalian cell systems (e.g., COS, CHO, BHK, 293 or 3T3 cells) harboring recombinant expression constructs containing promoters derived from the genome of mammalian cells (e.g., metallothionein promoter), mammalian viruses (e.g., the adenovirus late promoter or the vaccinia virus 7.5K promoter) or from artificial chromosome(s).

Fel d1 Polypeptide Fused to a Heterologous Carrier Protein.

In another embodiment, the immunogenic composition can contain a Fel d1 polypeptide conjugated to a carrier polypeptide, and a pharmaceutically-acceptable carrier.

The present invention also provides a method of administering to a cat an immunogenic composition comprising at least one Fel d1 polypeptide conjugated to a heterologous carrier polypeptide. A heterologous carrier polypeptide can be fused to the N-terminus or C-terminus of an antigenic polypeptide of the invention. Antigenic fusion proteins of the invention can be produced by techniques known to those of skill in the art, for example, by standard recombinant DNA techniques. For example, a nucleotide sequence encoding an antigenic fusion polypeptide can be synthesized by conventional techniques including automated DNA synthesizers. Alternatively, PCR amplification of nucleotide fragments can be carried out using anchor primers, which give rise to complementary overhangs between two consecutive nucleotide fragments, and which can subsequently be annealed and reamplified to generate a nucleotide sequence encoding an antigenic fusion polypeptide (see, e.g., Ausubel et al., eds., 1993 Current Protocols in Molecular Biology, John Wiley & Sons, NY). Moreover, many expression vectors are commercially available that already encode a fusion moiety (e.g., a GST polypeptide). A nucleic acid encoding an antigenic polypeptide of the invention can be cloned into such an expression vector such that the fusion moiety is linked in-frame to the antigenic polypeptide of the present invention. Further, a heterologous carrier polypeptide can be fused to an antigenic peptide by chemical methods known to those of skill in the art.

Alternatively, a recombinant Fel d1 polypeptide may be expressed as a fusion protein with rmLT.

2) Fel d1 Polynucleotides and Methods of Administration.

The present invention includes the administration of an immunogenic composition to a cat which comprises a polynucleotide molecule encoding at least one Fel d1 polypeptide, or fragment there of, or a viral vector containing a polynucleotide molecule encoding at least one Fel d1 polypeptide or fragment there of.

The composition may comprise either a polynucleic acid encoding a Fel d1 polypeptide, or a viral vector containing polynucleic acids encoding a Fel d1 polypeptide, or combinations of both.

The polynucleotides described here encode for Chain 1 with Leader A (SEQ ID NO. 1), Chain 1 with Leader B (SEQ ID NO. 3), Chain 2, Long form (SEQ ID NO. 5), Chain 2, Short form (SEQ ID NO. 7), or Chain 2, Short Truncated form (SEQ ID NO. 9), or fragments thereof.

Gene Therapy Methods

“Gene therapy” refers to therapy performed by the administration to a subject of an expressed or expressible nucleic acid. One or more nucleic acid molecules comprising a polynucleotide sequence encoding an antigenic peptide, or one or more nucleic acid molecules comprising a polynucleotide sequence encoding an antigenic peptide of the invention, and one or more nucleic acid molecules comprising a polynucleotide sequence encoding an antigenic fusion protein of the invention are administered by way of gene therapy.

In this embodiment of the invention, the nucleic acids produce their encoded antigenic peptides or antigenic fusion proteins that mediate a therapeutic effect by eliciting an immune response. Any of the methods for gene therapy available in the art can be used according to the present invention. For general reviews of the methods of gene therapy, see Goldspiel et al., 1993, Clinical Pharmacy 12: 488-505; Wu and Wu, 1991, Biotherapy 3: 87-95; and Mulligan, 1993, Science 260: 926-932. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In one embodiment, the polynucleotide encoding a Fel d1 polypeptide is amplified, ligated into an expression vector, and transformed into competent E. coli cells. A clone containing the insert in the proper orientation is identified by standard molecular biology techniques known to those skilled in the art. Bulk purified plasmid DNA for immunization is prepared by scaling up growth in approximately 10 liters of LB medium, isolating supercoiled plasmid DNA via CsCl density gradient centrifugation, followed by extensive dialysis. The purified DNA is dissolved in phosphate buffered saline (PBS) with 1 mM EDTA at a concentration of 2-5 μg/μl. Restriction digestion and endotoxin testing are performed for each plasmid DNA preparation. Experimental immunogenic compositions are prepared from the purified DNA. The appropriate volume of stock DNA from each construct is dissolved in sterile PBS to yield 300 μg DNA in a 2 mL dose. Placebo vaccine is also assembled using the vector DNA without insert. Immunogenic compositions can be administered via intramuscular injection. Or other routes of administration.

Viral Vectors

In another embodiment of the present invention, a viral vector that contains a polynucleotide encoding at least one Fel d1 polypeptide or antigenic fusion protein is prepared and administered to a cat. The polynucleotide encoding a Fel d1 polypeptide is cloned into a viral vector. Following transformation into competent E. coli cells and identification of an appropriate clone, the recombinant vector is purified. Mammalian cells are then transfected with the purified vector, and virus plaques are formed. A positive clone is identified by standard techniques known to those skilled in the art. Sufficient quantities of recombinant virus are then prepared by purifying virus particles from crude cell lysates and supernatants. The composition is administered to a host via intramuscular injection with approximately 109 virus particles.

In another embodiment, a retroviral vector containing nucleic acid sequences encoding a Fel d1 antigenic polypeptide or an antigenic fusion protein can be used (see, e.g., Miller et al., 1993, Meth. Enzymol. 217: 581-599). These retroviral vectors have been modified to delete retroviral sequences that are not necessary for packaging of the viral genome and integration into host cell DNA. The nucleic acid sequences encoding antigenic polypeptides or antigenic fusion proteins to be used in gene therapy are cloned into one or more vectors, which facilitates delivery of the gene into a patient. More detail about retroviral vectors can be found in Boesen et al., 1994, Biotherapy 6: 291-302. Adenoviruses are other viral vectors that can be used in gene therapy. Adenoviruses have the advantage of being capable of infecting non-dividing cells. Kozarsky and Wilson, 1993, Current Opinion in Genetics and Development 3: 499-503 present a review of adenovirus-based gene therapy. Adeno-associated virus (AAV) has also been proposed for use in gene therapy (see, e.g, Walsh et al., 1993, Proc. Soc. Exp. Biol. Med. 204: 289-300; and U.S. Pat. No. 5,436,146).

In an alternate embodiment, Fel d1 is delivered to a cat via a prime-boost method, comprising first administering to the cat a priming immunogenic composition comprising nucleic acid molecule(s) encoding and expressing in vivo Fel d1 protein, a fragment(s) thereof or epitope(s). Thereafter, a boosting immunogenic composition that presents to the cat's immune system the same protein, fragment, or epitope, is administered. The boosting immunogenic composition is advantageously different than the nucleic acid immunogenic composition. For instance, the boosting composition can be Fel d1 protein, fragments or epitopes, or a recombinant or modified vector, e.g., viral. A recombinant or modified vector is advantageously an in vivo expression vector, such as a modified or recombinant bacteria, yeast, virus, e.g. poxvirus, adenovirus or herpesvirus, comprising nucleic acid molecule(s) encoding and expressing in vivo the Fel d1 protein, fragments or epitopes expressed by the nucleic acid-based immunogenic composition. The boost is advantageously performed with Fel d1 protein, fragments or epitopes, or with an immunogenic composition comprising a recombinant live viral vector, including but not limited to a recombinant poxvirus, adenovirus, or herpesvirus, that comprises a nucleic acid molecule(s) encoding and expressing in vivo the Fel d1 protein, fragment(s) or epitope(s) expressed by the nucleic acid-based immunogenic composition. Thus, it is advantageous that the boost either comprises the protein, fragment or epitope expressed by the nucleic acid-based immunogenic composition, or expresses in vivo the same protein, fragment or epitope expressed by the nucleic acid-based immunogenic composition.

3) Methods of Administration of Fel d1 Peptides and Polynucleotides to Cats.

The treatments provided here provide for a technique that allows the direct reduction of allergens present on cats, and consequentially reducing the allergic responsiveness of sensitized mammals, especially humans. That is, the administration of native or appropriately modified Fel d1 to a cat provides a method of treating sensitivity in a sensitized individual mammal including humans, but also companion animals, including dogs and cats. Self allergic reactions of cats to cat allergen can also be reduced. Diseases such as eosinophilic granuloma can be treated with the administrations of Fel d1 described herein. Dogs can also be sensitized to cat dander and thus treated with the administrations of Fel d1 described herein. Humans suffer from being sensitive to cat dander, in particular Fel d1, and they can thus be treated or helped by the administrations of Fel d1 described herein. Any sensitive animal exposed to the cat habitat could experience a reduced allergic response and benefit from a reduction in Fel d1 from that place of habitat and from a reduction of Fel d1 from any cats proximate to the human or sensitized animal. We describe the administering to a cat of an immunogenic composition comprising Fel d1, including a polypeptide, a polynucleic acid encoding a Fel d1 polypeptide, or a viral vector encoding a Fel d1 polypeptide.

Frequency of Administration

The immunogenic compositions described herein can be administered to a cat once, or more than once, such that it produces a full, broad immunogenic response and or achieves a reduction in the amount of Fel d1 shed. In one embodiment of the present invention, the cat is subjected to a series of administrations to produce a full, broad immune response. When provided in a series, the administrations can be provided between about one day to four weeks, or longer, apart. In particular embodiments, the composition can be administered to a cat at different sites simultaneously. Fel d1 may be administered once, or once with a booster in from 2 days to 2 months, or in about two weeks, or it may be administered once and then followed with an annual or semi-annual booster, or any combination thereof.

The immunogenic composition of the present invention is administered and dosed in accordance with good medical practice, taking into account the clinical condition of the individual subject, the site and method of administration, scheduling of administration, subject age, sex, body weight and other factors known to medical practitioners.

Routes of Administration

The mode of administration of the immunogenic compositions of the present invention can be any suitable route that delivers the composition to the cat. The immunogenic composition may be administered parenterally including by injection, subcutaneously and intramuscularly, intradermal, transdermal, intraperitoneal, intravenously, and scarification (scratching through the top layers of skin, e.g., using a bifurcated needle). The compositions can be administered orally, including intranasal, oronasal, oral, intraocular, such as by nose, mouth or eye with liquids, drops, sprays, inhalers or powders.

Pharmaceutically-Acceptable Carriers

Fel d1 polypeptide can be administered either alone or in association with one or more pharmaceutically-acceptable carrier. “In association with” can refer to components in the same or separate containers and can refer to solutions, mixtures, suspensions or other combinations of ingredients and it may refer to ingredients that do or do not associate chemically, that is they may or may not chemically interact or form any type of chemical bonds with each other.

The immunogenic composition for any one of the embodiments of the present invention is formulated in a pharmaceutically accepted carrier according to the mode of administration to be used. Such carriers include any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers include albumin, among others. Adjuvants include, but are not limited to, the RIBI adjuvant system (Ribi Immunochem Research, Inc.; Hamilton, Mont.), alum, aluminum hydroxide gel, Alhydrogel, oil-in water emulsions, water-in-oil emulsions such as, e.g., Freund's complete and incomplete adjuvants, Block co polymer (CytRx; Atlanta, Ga.), DEAE-Dextran, AbiSCO, ImS2212VG (Seppic, France), SAF-M (Chiron; Emeryville, Calif.), AMPHIGEN® adjuvant, saponin, Quil A, QS-21 (Cambridge Biotech Inc.; Cambridge, Mass.), or other saponin fractions, monophosphoryl lipid A, pyridine lipid-amine adjuvant, cholera toxin, muramyl dipeptide, cationic or anionic polymers, synthetic constructs, ISCOMS, mineral salts, Mycobacterial, bacterial and plant derivatives, surface-active agents or microparticles, among others. The immunogenic compositions can further include one or more other immunomodulatory agents, such as interleukins, interferons, other cytokines or Toll receptor agonists.

In another embodiment, the immunogenic composition may comprise Fel d1 polypeptide administered together with recombinant mutant labile toxin (rmLT) from E. coli (Dickinson, B. L., and Clements, J. D.; Infect. Immun. 63(5):1617-1623; (1995). Recombinant Fel d1 or Fel d1-neutralizing epitopes or polypeptides can also be administered with non-replicating, non-infectious, but highly immunogenic virus-like particles or VLPs. See US 20040005338, section [0019]. VLPs are being exploited in the area of vaccine production because of both their structural properties and their non-infectious nature. See WO 98/50071. VLPs are super molecular structures built in a symmetric manner from many protein molecules of one or more types. They lack the viral genome and, therefore, are noninfectious. VLPs can often be produced in large quantities by heterologous expression and can be easily purified. The immunogenic composition may be encapsulated for controlled release and/or controlled delivery, such as with mucosal delivery to targeted enteric locations. Encapsulation vehicles include, but are not limited to, lipid containing vesicles, biodegradable polymer microspheres and emulsions.

Immunogenic protein carriers also include, but are not limited to, intact Keyhole Limpet Hemocyanin (KLH), subunit KLH, or Diptheria Toxoid (DT).

The vector used for administration may be in the form of a live virus. A live virus vector may be used according to procedures known in the art.

Form of the Compositions

The immunogenic compositions of the present invention can be made in various forms depending upon the route of administration. For example, the immunogenic compositions can be made in the form of sterile aqueous solutions or dispersions suitable for injectable use, or made in lyophilized forms using freeze-drying techniques. Lyophilized immunogenic compositions are typically maintained at about 4° C., and can be reconstituted in a stabilizing solution, e.g., saline or HEPES, with or without adjuvant.

Amount to be Administered

For purposes of this invention, an immunogenic amount, when administered, comprises about 0.1 μg-1 mg of purified protein, 0.1 μg-10 mg of nucleic acid, or for immunogenic compositions containing virus, an effective amount will generally range from about 10⁵ TCID₅₀ to about 10⁸ TCID₅₀, inclusive. “TCID₅₀” refers to “tissue culture infective dose”, and is defined as that dilution of a virus required to infect 50% of a given batch of inoculated cell cultures. In a formulation containing multiple components, the same or lesser immunogenic amounts can usefully be employed.

Appropriate therapeutically effective doses can be determined readily by those of skill in the art based on the above immunogenic amounts, the condition being treated and the physiological characteristics of the animal. Accordingly, an immunogenic composition provides a dosage of a sterile preparation of an immunogenic amount of the active ingredient(s), where the active ingredient is at least one bacteria, protein, nucleic acid, or any combination thereof. In the presence of additional active agents, these unit dosages can be readily adjusted by those of skill in the art.

4) Methods of Reducing Allergic Responsiveness in Humans to Cat Dander Comprising Administration of Monoclonal Antibodies to Fel d1 and Administration of Fel d1 RNA to Cats.

The present invention may also include other therapeutic methods for reducing the amount of Fel d1 shed by a cat.

Antibodies

This invention includes the use of monoclonal antibodies delivered to a cat. It includes delivered by spray, wiping, dipping, rubbing or other topical administration technology to a cat. Alternatively, the antibodies could be injected into the host. Following injection, the antibodies would bind with the self-protein, thus activating the immune system to eliminate the protein. The antibodies could also bind to the Fel d1 in such a manner so as to block epitopes recognized by human IgE, thus eliminating or diminishing the allergic response in humans.

RNA Silencing

The present invention could also include RNA silencing (siRNA), in which long double-stranded RNAs (dsRNAs), typically >200 nucleotides, are used to silence expression of the fel d1 chain 1 and/or chain 2 genes. Therapeutic delivery of the fel d1 siRNA could be by viral vector delivered parenterally, or targeted delivery to the sebaceous gland cells via endosomes. Another method included would be the use of fel d1 antisense RNA to reduce the amount of Fel d1 shed. As it is possible for RNA to form duplexes similar to DNA duplexes, delivery of a sequence of RNA (the “antisense RNA”) complementary to the messenger strand of RNA (mRNA) can lead to formation of double stranded RNA, which results in inhibition of gene expression.

Advantages and Utility for the Invention

The present invention, unlike other treatments and therapies, provides for the allergen-derived immunogenic composition to be administered to a cat, and not to a sensitized individual. The invention directly reduces the amount of antigen sensitized individuals in the individuals' environment.

The present invention is completely safe for humans, particularly those who are highly sensitized to the allergen, including infants and young children. Unknown consequences might occur later in life with respect to development of the immune system when exposed to drugs and desensitization treatments. (Prescott and Jones, 2002; Curr. Drug Targets-Inflammat. & Allergy 1:65-75).

Another advantage of the present invention is that in reducing the amount of Fel d1 shed by a single cat, a beneficial effect is realized by everyone sensitized to the allergen that comes into contact with the cat. The present method is directed at the host directly responsible for production of the allergen. Reducing the environmental load of Fel d1 generated by a cat or cats can have a significant positive impact in areas where multiple individuals are exposed to that animal or where multiple animals are exposed to sensitized individuals.

Another advantage of the present invention is that it represents a way to treat animals presently in environments where sensitized individuals are exposed. There are currently no available options to cat owners in this situation except frequent washing of the cat and removal of the cat. This invention could allow animals to stay in the same environment with sensitized mammals, including humans, while treatments were ongoing.

Another advantage of the present invention is that although methods are in place for manipulation of the genome of cats to disrupt the sequence encoding Fel d1, such methods are not applicable to cats already in existence. Therefore, the present invention provides a method that would be available to those already owning cats.

### SPECIFIC EXAMPLES OF THE INVENTION

The examples described below are not intended to be limiting in any way. One skilled in the art is expected to use the entire description of the invention provided here, including the examples below to fully understand, enable, and have in mind a complete description of the invention in all of its many forms and possibilities.

### Example 1 Antigen Preparation

Recombinant Fel d1 (rFel d1) is commercially available from INDOOR Biotechnologies (Charlottesville, Va.). The recombinant protein is a fusion composed of a 15-residue linker between chains 1 and 2 with a C-terminal His tag. The linker, along with expression in Pichia pastoris, ensures properly folded, processed and glycosylated protein. The recombinant protein has similar folding as the native Fel d1 (nFel d1), and has been shown to have indistinguishable immunologic properties compared to the native as measured by human IgE binding studies. rFel d1 is sterile, has no greater than 0.5 EU/ug of endotoxin, and is available at a concentration of 1-2 mg/ml.

In order to make Fel d1 (a “self” protein) antigenic, the carrier protein Keyhole Limpet Hemocyanin (KLH, whole or subunit), purchased from Stellar Biotechnologies (Port Hueneme, Calif.), was chemically conjugated to rFel d1, the objective being to elicit a Fel d1 antibody response in the cat. Conjugation was conducted by standard crosslinking chemistry using gluteraldehyde, although other crosslinking agents may also be used, e.g., maleimide, 1-Ethyl-3(3-dimethylaminopropyl) carbodiimide HCL Conjugation is possible with another antigenic carrier protein, Diptheria Toxoid (DT). Characterization assays and filterability were employed to select one carrier-chemistry combination to proceed to scale-up for proof-of-concept (POC) study antigen.

Subunit KLH (approx. 400 KD) has a significantly lower molecular weight than the intact KLH (8-9 million daltons). The subunit form was selected for initial testing due to better ease-of-use, although the whole protein is also expected to be immunogenic. Glutaraldehyde is homobifunctional, crosslinking proteins at N-terminal amino groups, plus certain lysine 1-amino groups (pKa-dependent). EDC is heterobifunctional, binding to amino and carboxylate groups. M-Maleimideobenzoic acid N-hydroxysuccinimide (MBS or “maleimide”) is heterobifunctional, linking to primary amines and sulfhydryl groups. The advantage of the maleimide reaction is that it is fast, selective, and proteins are available pre-activated. One advantage to using glutaraldehyde is that it is the most commonly used crosslinker, thus numerous protocols are available.

The recombinant protein conjugated to the subunit intact KLH was filtered through a 0.2 micron filter. The conjugated rFel d1 can be formulated with a variety of different adjuvants. Immunogenic composition groups were formulated following conjugation of rFel d1 with carrier protein. The following adjuvants were formulated with rFel d1-carrier: 1) Adjuvant combination; 2) DEAE-Dextran; 3) Glycolipid Bay R1005; 4) AbISCO; and 6) Amphigen. Additional back-up adjuvants include Rehydrogel, Pertussis whole cell adjuvant, “Cat Cocktail”+Bay R1005, ImS2212VG, Iscomatrix and Toll-like receptor 7 agonist. The formulated immunogenic compositions were confirmed sterile prior to their use in the efficacy study.

### Example 2 Measuring Fel d1 Concentration in Cat Hair and Saliva Extracts with ELISA

Extracted-hair and saliva samples were analyzed for Fel d1 protein concentration measurement as determined by ELISA (INDOOR Biotechnologies). Anti-Fel d1 monoclonal antibody (mAb) 6F9 was supplied HPLC-purified as a stock solution at 1 mg/ml in PBS. The antibody was diluted 1/1000 (i.e. 10 μl/10 ml) in 50 mM carbonate-bicarbonate buffer, pH 9.6. Polystyrene NUNC microtiter plates (Fisher Scientific International Inc.; Hampton, N.H.) were coated with 100 μl per well of the diluted mAb 6F9, and incubated overnight at 4° C. The wells were then washed 3× with PBS-0.05% Tween 20, pH 7.4 (PBS-T), 100 μl 1% BSA PBS-T was added per well, and the plates were incubated for 30 min at room temperature. The plates were then washed 3× with PBS-T, 100 μl of Fel d1 allergen standard was added to each well, and the plate was incubated for 1 hr at room temperature. A Fel d1 control curve was generated using doubling dilutions of the allergen standard. The control curve dilutions were from 80-0.16 ng/ml Fel d1. 20 μl of Fel d1 standard was pipetted into 180 μl 1% BSA PBS-T placed in wells A1 and B1 of the ELISA plate, and mixed well. 100 μl was then transferred across the plate into 100 μl 1% BSA PBS-T diluent to make 10 serial doubling dilutions. Wells A11, B11, A12 and B12 contained only 1% BSA PBS-T as blanks. Wells were washed 3× with PBS-T, and 100 μl diluted biotinylated anti-Fel d1 mAb 3E4 was added to each well. The antibody solution contained 50% glycerol, and was diluted 1/1000 (i.e. 10 μl/10 ml) in 1% BSA-PBS-T. Plates were then incubated for 1 hr at room temperature. Wells were then washed 3× with PBS-T, and 100 μl of diluted Streptavidin—Peroxidase (0.25 mg reconstituted in 1 ml distilled water; obtained from Sigma-Aldrich, St. Louis, Mo.;) was added to each well. The reconstituted Streptavidin was diluted 1/1000 (i.e. 10 μl/10 ml) in 1% BSA PBS-T. Plates were incubated for 30 minutes at room temperature. Wells were then washed 3× with PBS-T, and 100 μl 1 mM ABTS substrate in 70 mM citrate phosphate buffer, pH 4.2 containing a 1/1000 dilution of 30% H₂O₂ (i.e. 10 μl/10 ml ABTS) was added to each well. The plates were read until the optical density at 405 nm reached 2.0-2.4.

### Example 3 Efficacy Testing of Fel d1 Immunogenic Compositions

The ability of rFel d1-based immunogenic compositions, formulated with various adjuvants and administered orally or patenterally, to elicit Fel d1 antibodies in cats and reduce shedding of the allergen will be assessed. Because washing a cat reduces the amount of Fel d1 shed, washed cats will be used as positive controls for allergen reduction in the POC study. The positive control cats will be washed once per week. Eight to ten cats per composition group will allow for a measurement sensitivity of down to 15% reduction in Fel d1 concentrations below that in the untreated group. Seven different compositions will be administered (Table 1), including:

### 1) Saline

### 2) Positive Control for Fel d1 reduction (washed cats)

3) rFel d1-CARRIER conjugate+adjuvant combination
4) rFel d1-CARRIER conjugate+DEAE-Dextran
5) rFel d1-CARRIER conjugate+Glycolipid Bay R1005
6) rFel d1-CARRIER conjugate+AbISCO
7) rFel d1-CARRIER conjugate+Amphigen
Additional “back-up” treatment groups are conjugated rFel d1 with Alhydrogel, Pertussis whole cell, “cat cocktail”+Bay R1005, and Toll-like receptor 7 agonist. The initial test carrier is subunit KLH, with whole KLH and DT as backup antigenic carrier proteins. Eighty (80) adult, purpose-bred cats will be utilized in this study. All animals will be in good general health. Male and female, neutered or intact, cats will be enrolled. Each animal will be identified with a unique ear tattoo. Animals will be individually housed. Space (including feeder space) for the animals will meet or exceed requirements as set forth in 9 CFR and the Guide for the Care and Use of Laboratory Animals. Feed will be consistent with standard practices of the testing facility. Diet will be ad libitum dry cat food suitable for the age and nutritional requirements of the animals. Water from the municipal water supply will be provided ad libitum at all times during the study.

TABLE 1
Investigational Veterinary Product (IVP)

| | | | Number | |
|---|---|---|---|---|
| | Antigen | Adjuvant Type and | of | Volume |
| IVP | Dose | Amount per Dose | Doses | per Dose |
| Immunogenic | 50 ug | Adjuvant | 66 | 1 ml |
| Composition | | combination ug | | |
| #1 | | | | |
| Immunogenic | 50 ug | DEAE-Dextran, | 66 | 1 ml |
| Composition | | 2% w/v or 20 mg/ | | |
| #2 | | dose | | |
| Immunogenic | 50 ug | Glycolipid Bay | 66 | 1 ml |
| Composition | | R1005, | | |
| #3 | | 1 mg/ml | | |
| Immunogenic | 50 ug | AbISCO, | 66 | 1 ml |
| Composition | | 0.1 mg/dose | | |
| #4 | | | | |
| Immunogenic | 50 ug | Amphigen, | 66 | 1 ml |
| Composition | | 1% | | |
| #5 | | | | |
| Immunogenic | 50 ug | ImS221VG | 66 | 1 ml |
| Composition | | 15% v/v | | |
| #6 | | | | |
| Immunogenic | 50 ug | Alhydrogel | 66 | 1 ml |
| Composition | | 1% v/v | | |
| #7 | | | | |
| Immunogenic | 50 ug | Adjuvant | 66 | 1 ml |
| Composition | | Combination | | |
| #8 | | 1 mg/ml + R1005 | | |

Animals will be allocated to treatment groups, rooms and pens according to randomization plans. Personnel making animal observations will be unaware of individual treatments, with the exception that animals in T02 will be washed weekly (Table 2). Hair and serum samples will be collected approximately weekly. All hair samples will be labeled in such a way as to make laboratory personnel unaware of treatment group or animal of origin.

TABLE 2
Experimental Design

| | Investigational | | Approximate study days (+/−1 day) | | | |
|---|---|---|---|---|---|---|
| | Veterinary | | Subcutaneous | Sample | | |
| Group | Product (IVP) | N | Injection | collection | Clinical obs. | Wash |
| T01 | Saline (positive | 10 | Day 0, D21, | Day 0, D7, | Day 0, D7, | N/A |
| | control) | | D42 +/− D63 +/− D84 +/− D105 | D14, D21. | D14, D21. | |
| | | | | D28. D35, | D28. D35, | |
| | | | | D42, D49, | D42, D49, | |
| | | | | D56, +/−(D63, | D56, +/−(D63, | |
| | | | | D70, | D70, | |
| | | | | D77, D84, | D77, D84, | |
| | | | | D91, D98, | D91, D98, | |
| | | | | D105, D112, | D105, D112, | |
| | | | | D119) | D119) | |
| T02 | Saline | 10 | D0, D21, D42 +/− D63 +/− D84 +/− D105 | D0, D7, | D0, D7, D14, | Day 0, D7, |
| | (washed- | | | D14, D21. | D21. D28. | D14, D21. |
| | negative | | | D28. D35, | D35, D42, | D28. D35, |
| | control) | | | D42, D49, | D49, D56, +/−(D63, | D42, D49, |
| | | | | D56, +/−(D63, | D70, D77, | D56, +/−(D63, |
| | | | | D70, | D84, D91, | D70, D77, |
| | | | | D77, D84, | D98, D105, | D84, D91, |
| | | | | D91, D98, | D112, D119) | D98, |
| | | | | D105, D112, | | D105, |
| | | | | D119) | | D112, |
| | | | | | | D119) |
| T03 | Immunogenic | 10 | D0, D21, D42 +/− D63 +/− D84 +/− D105 | D0, D7, | D0, D7, D14, | N/A |
| | Composition | | | D14, D21. | D21. D28. | |
| | #1 | | | D28. D35, | D35, D42, | |
| | | | | D42, D49, | D49, D56, +/−(D63, | |
| | | | | D56, +/−(D63, | D70, D77, | |
| | | | | D70, | D84, D91, | |
| | | | | D77, D84, | D98, D105, | |
| | | | | D91, D98, | D112, D119) | |
| | | | | D105, D112, | | |
| | | | | D119) | | |
| T04 | Immunogenic | 10 | D0, D21, D42 +/− D63 +/− D84 +/− D105 | D0, D7, | D0, D7, D14, | N/A |
| | Composition | | | D14, D21. | D21. D28. | |
| | #2 | | | D28. D35, | D35, D42, | |
| | | | | D42, D49, | D49, D56, +/−(D63, | |
| | | | | D56, +/−(D63, | D70, D77, | |
| | | | | D70, | D84, D91, | |
| | | | | D77, D84, | D98, D105, | |
| | | | | D91, D98, | D112, D119) | |
| | | | | D105, D112, | | |
| | | | | D119) | | |
| T05 | Immunogenic | 10 | D0, D21, D42 +/− D63 +/− D84 +/− D105 | D0, D7, | D0, D7, D14, | N/A |
| | Composition | | | D14, D21. | D21. D28. | |
| | #3 | | | D28. D35, | D35, D42, | |
| | | | | D42, D49, | D49, D56, +/−(D63, | |
| | | | | D56, +/−(D63, | D70, D77, | |
| | | | | D70, | D84, D91, | |
| | | | | D77, D84, | D98, D105, | |
| | | | | D91, D98, | D112, D119) | |
| | | | | D105, D112, | | |
| | | | | D119) | | |
| T06 | Immunogenic | 10 | D0, D21, D42 +/− D63 +/− D84 +/− D105 | D0, D7, | D0, D7, D14, | N/A |
| | Composition | | | D14, D21. | D21. D28. | |
| | #4 | | | D28. D35, | D35, D42, | |
| | | | | D42, D49, | D49, D56, +/−(D63, | |
| | | | | D56, +/−(D63, | D70, D77, | |
| | | | | D70, | D84, D91, | |
| | | | | D77, D84, | D98, D105, | |
| | | | | D91, D98, | D112, D119) | |
| | | | | D105, D112, | | |
| | | | | D119) | | |
| T07 | Immunogenic | 10 | D0, D21, D42 +/− D63 +/− D84 +/− D105 | D0, D7, | D0, D7, D14, | N/A |
| | Composition | | | D14, D21. | D21. D28. | |
| | #5 | | | D28. D35, | D35, D42, | |
| | | | | D42, D49, | D49, D56, +/−(D63, | |
| | | | | D56, +/−(D63, | D70, D77, | |
| | | | | D70, | D84, D91, | |
| | | | | D77, D84, | D98, D105, | |
| | | | | D91, D98, | D112, D119) | |
| | | | | D105, D112, | | |
| | | | | D119) | | |
| T08 | Immunogenic | 10 | D0, D21, D42 +/− D63 +/− D84 +/− D105 | D0, D7, | D0, D7, D14, | N/A |
| | Composition | | | D14, D21. | D21. D28. | |
| | #6 | | | D28. D35, | D35, D42, | |
| | | | | D42, D49, | D49, D56, +/−(D63, | |
| | | | | D56, +/−(D63, | D70, D77, | |
| | | | | D70, | D84, D91, | |
| | | | | D77, D84, | D98, D105, | |
| | | | | D91, D98, | D112, D119) | |
| | | | | D105, D112, | | |
| | | | | D119) | | |

Commercially available sterile saline (0.9% NaCl solution) for injection will be used in groups T01 and T02. Experimental immunogenic compositions passed a sterility test prior to initiation of the study. As Fel d1 is a naturally-occurring peptide secreted in varying amounts under normal conditions by cats, no challenge will be administered as a part of this study.

Animals will be allowed to acclimate in their housing for a minimum of seven days prior to the first injection. Cats will be injected with the appropriate immunogenic composition on study day 0 in the intrascapular region. (Should pathology such as swelling or pain in the intrascapular area present prior to administration of any immunogenic composition, another appropriate site will be selected by study personnel and noted.) Personnel administering immunogenic compositions will be masked to treatment. However, the investigator will be responsible for ensuring each animal receives the correct immunogenic composition. Completeness of immunogenic composition administration will be documented. An assessment of each animal's reaction (vocalization, scratching or escape attempt) will be recorded. This procedure will be repeated on study days 21 and 42, and possibly on days 63, 84 and 105. (Whether animals will receive the immunogenic composition on days 63, 84 and 105 will be determined by study results.) A decision to continue administration of the immunogenic compositions will be made and communicated to the investigator during the week prior to day 63.

Prior to administration of each immunogenic composition, the animal's rectal temperature will be recorded, as well as on the day following each administration. If an animal has a rectal temperature above 103.7° F. on the day following administration of the composition, that animal will have its rectal temperature recorded daily until it is lower than 103.7° F. Additionally, on the day following administration of the composition, each animal will have its administration site examined for swelling, heat and pain. If pathology is present, approximate measurements of length and width will be recorded. In addition, animals will be observed daily for abnormal clinical signs (i.e. lethargy, reluctance to eat, generalized pain, etc.), and those observations recorded.

On day 0, prior to administration of the immunogenic composition, and weekly (days 0, 7, 14, 21, 38, 35, 42, 49, 56 63 and 70, when applicable) prior to administration, personnel will pluck approximately 10 mg hair from the nape of the neck (equidistant between and approximately 3 inches distal to a line running between the distal terminus of the ear pinna) of each cat. 10 mg of hair is equal to approximately 40 hairs, and should not cause the animal distress. Hair will be placed in labeled bags according to the masking protocol. If the decision is made to inject any or all animals on study days 63, 84, and 105, collection of hair samples will continue on days 77, 84, 91, 98, 105, 112, and 119, using the procedures described. Additionally, on day 0 prior to administration of the immunogenic composition, and weekly (days 0, 7, 14, 21, 38, 35, 42, 49, 56 63 and 70, when applicable) prior to administration, personnel will collect 3-5 mls of whole blood into a serum separator tube from each cat. Serum will be separated from the whole blood according to facility procedures, decanted into pre-labeled cryovial tubes, and frozen until analyzed.

The hair collected will be divided into duplicate 5 mg samples. Saliva will be collected on swabs. Protein contained in each sample will then be extracted with a 1 ml buffer solution, and the extract sent to INDOOR Biotechnologies for Fel d1 quantification. Serum samples will be analyzed by ELISA or Western blotting. (see Example 2 for Fel d1 concentration ELISA Protocol.)

Quantities of Fel d1 extracted from hair samples will be the primary variable measured in assessing efficacy of the various immunogenic compositions. Criteria for a valid test will be based upon whether animals in group T02 have significantly lower amounts of Fel d1 extracted from hair samples than animals in T01. A statistically significant reduction in the level of Fel d1 protein in hair extracts of vaccinated cats compared to negative controls is desired. If animals in treatment group T03, T04, T05, T06, T07 or T08 have lower Fel d1 concentrations than animals in group T01, that immunogenic composition will be considered to have directly contributed to a reduction in shedding of Fel d1 in treated animals. Additional supporting data would be a reduction in Fel d1 levels comparable to that of the positive control (washed) cats. Detection of antibody to Fel d1 in vaccinated cat sera by ELISA or Western blotting will indicate a serologic response to the immunogenic composition. (see Example X for serology ELISA Protocol.).

### Example 4 Measuring Antibody to Fel d1 in Cat Serum by ELISA

An ELISA for measuring Fel d1 antibodies in cat sera to confirm serologic response to immunogenic compositions will be developed, optimized and validated. rFel d1 and/or nFel d1 will be used as the capture antigen. Initially, rabbit polyclonal antibody will be used to screen for a positive control primary antibody (cat anti-Fel d1). The assay will then be developed and validated.

Although the present invention has been described in considerable detail with reference to certain preferred versions thereof, other versions are possible. Therefore, the scope of the appended claims should not be limited to the description of the preferred versions contained herein.

What is claimed is:

1. A method of reducing the amount of Fel d1 shed by a cat comprising:
administering to a cat an immunogenic composition comprising at least one Fel d1 polypeptide or fragment thereof.

2. The method of claim 1, wherein said composition comprises at least one recombinantly-produced Fel d1 polypeptide or fragment thereof, and a pharmaceutically-acceptable carrier.

3. The method of claim 2, wherein said Fel d1 polypeptide is a fusion comprising chain 1 and chain 2 of Fed d1 linked with a 15 residue linker between chains 1 and 2 with a C-terminal His tag.

4. The method of claim 3, wherein said Fel d1 polypeptide is conjugated to subunit a Keyhole Limpet Hemocyanin carrier protein.

5. The method of claim 1, wherein said composition comprises at least one naturally-occurring Fel d1 polypeptide or fragment thereof, and a pharmaceutically-acceptable carrier.

6. The method of claim 1, wherein said composition further comprises at least one Fel d1 polypeptide or fragment thereof conjugated to a heterologous carrier polypeptide, and a pharmaceutically-acceptable carrier.

7. The method of claim 1, wherein said composition further comprises at least one Fel d1 polypeptide or fragment thereof associated with a virus-like particle, and a pharmaceutically-acceptable carrier.

8. A method of reducing the amount of Fel d1 shed by a cat comprising: administering to a cat an immunogenic composition comprising a polynucleotide molecule encoding at least one Fel d1 polypeptide or fragment thereof.

9. A method of claim 6 comprising: administering to a cat an immunogenic composition comprising a viral vector containing a polynucleotide molecule encoding at least one Fel d1 polypeptide or fragment thereof.

10. A method of reducing the amount of Fel d1 shed by a cat comprising: administering to a cat an immunogenic composition selected from the group consisting of monoclonal antibodies or siRNA.

11. The method of any of claim 1, wherein said composition is administered to a cat at least once.

12. The method of any of claim 1, wherein said composition is administered orally.

13. The method of any of claims 1, wherein said composition is administered by parenteral injection, subcutaneous injection or intramuscular injection.

14. A method of treating mammals who are allergic to cats comprising: administering to a cat an immunogenic composition comprising Fel d1.

15. A method of claim 14 where said mammal is a human, cat, dog or horse.

16. A method of treating sensitivity in a mammal to Fel d1 comprising: administering to a cat an immunogenic composition comprising at least one Fel d1 polypeptide or fragment thereof, a polynucleotide molecule encoding at least one Fel d1 polypeptide or fragment thereof, or a viral vector encoding at least one Fel d1 polypeptide or fragment thereof.

17. The method of claim 16, wherein said immunogenic composition comprises at least one Fel d1 polypeptide or fragment thereof.
